(19) United States
(12) Patent Application Publication
(10) Pub. No.: US 2024/0415432 A1
(43) Pub. Date: Dec. 19, 2024

(54) CATHETER SYSTEM FOR ARTERIAL BLOOD DRAW AND RELATED METHODS

(71) Applicant: Becton, Dickinson and Company, Franklin Lakes, NJ (US)

(72) Inventors: Yiping Ma, Layton, UT (US); Jonathan Karl Burkholz, Salt Lake City, UT (US); Christopher Anthony Basciano, Apex, NC (US); Sivaramakrishnan Balasubramanian, Cary, NC (US); Siddharth Nagarajan, Bangalore (IN)

(21) Appl. No.: 18/677,619

(22) Filed: May 29, 2024

Related U.S. Application Data
(60) Provisional application No. 63/521,027, filed on Jun. 14, 2023.

(51) Int. Cl. A61B 5/15; A61M 25/00

(52) U.S. Cl. CPC A61B 5/150992; A61M 25/0014

(57) ABSTRACT

An arterial catheter system may include a catheter assembly, which may include a catheter hub and an arterial catheter. The arterial catheter system may include a needle assembly, which may include a needle hub and an introducer needle. The arterial catheter system may include an extension tube coupled to the catheter assembly and a fluid pathway extending through the arterial catheter, catheter hub, and the extension tube. A first fluidic resistance within a portion of the fluid pathway within the extension tube may be greater than a second fluidic resistance within the fluid pathway distal to or proximal to the portion of the fluid pathway. The extension tube may have a length L and an inner diameter D. A geometric factor G_{f} of the portion of the fluid pathway may equal L/D⁴ and may be selected to reduce hemolysis during blood draw from an artery.

### RELATED APPLICATIONS

This application claims the benefit of U.S. Provisional Patent Application No. 63/521,027, which was filed on Jun. 14, 2023, which is incorporated herein in its entirety.

### BACKGROUND

A catheter is commonly used to infuse fluids into vasculature of a patient. For example, the catheter may be used for infusing normal saline solution, various medicaments, or total parenteral nutrition. The catheter may also be used for withdrawing blood from the patient.

The catheter may include an over-the-needle peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe or a test tube with a rubber stopper at one end. In some instances, the blood collection container has had all or a portion of air removed from the test tube so pressure within the blood collection container is lower than ambient pressure. Such a blood collection container is often referred to as an internal vacuum or a vacuum tube. A commonly used blood collection container is a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells are in a high shear stress state and susceptible to hemolysis due to a high initial pressure differential between the vein and the blood collection container. Hemolysis may result in rejection and discard of a blood sample. The high initial pressure differential can also result in catheter tip collapse, vein collapse, or other complications that prevent or restrict blood from filling the blood collection container. As the blood collection container fills, a pressure differential between the vein and the blood collection container decreases, and filling of the blood collection tube with blood slows significantly.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure generally relates to an arterial catheter system, as well as related devices, systems, and methods. In some embodiments, the arterial catheter system may include a catheter assembly, which may include a catheter hub. In some embodiments, the catheter hub may include a distal end and a proximal end. In some embodiments, the catheter assembly may include an arterial catheter extending from the distal end of the catheter hub. In some embodiments, the arterial catheter may be shorter and/or more rigid than an intravenous catheter, such as a peripheral intravenous catheter.

In some embodiments, the arterial catheter system may include a needle assembly, which may include a needle hub and an introducer needle. In some embodiments, the arterial catheter system may include an extension tube coupled to the catheter assembly and having a distal end and a proximal end. In some embodiments, the arterial catheter system may include a fluid pathway extending through the arterial catheter, catheter hub, and the extension tube.

In some embodiments, a geometric factor G_{f} of a portion of the fluid pathway within the extension tube may equal L/D⁴, wherein L is the length of the extension tube and D is the inner diameter of the portion of the fluid pathway within the extension tube. In some embodiments, the geometric factor G_{f} of the portion of the fluid pathway within the extension tube may be defined such that a fluidic resistance

$${R}_{f} = \frac{128 μ}{π} {G}_{f} ,$$

where

$${G}_{f} = \frac{L}{{D}^{4}} .$$

in some embodiments, the geometric factor G_{f} of the portion of the fluid pathway may be selected to reduce a maximum shear stress on arterial blood drawn from an artery and thereby reduce a risk of hemolysis of the arterial blood.

In some embodiments, the fluidic resistance may be a first fluidic resistance. In some embodiments, the first fluidic resistance of the portion of the fluid pathway within the extension tube may be greater than a second fluidic resistance within the fluid pathway distal to or proximal to the portion of the fluid pathway. In some embodiments, the second fluidic resistance may be defined in a manner similar to the first fluidic resistance.

In some embodiments, the first fluidic resistance, which may be lower than the second fluidic resistance, may facilitate a decreased flow rate of arterial blood within the portion of the fluid pathway within the extension tube such that the maximum shear stress is reduced within the portion of the fluid pathway and there is a decreased risk of hemolysis of the arterial blood that will be collected. In these embodiments, the length L and the inner diameter D of the extension tube, which may determine the geometric factor G_{f} of the portion of the fluid pathway, may be selected to increase the first fluidic resistance and decrease the flow rate within the portion of the fluid pathway such that the risk of hemolysis is decreased but the flow rate is still adequate for blood collection.

In some embodiments, the extension tube may be a first extension tube, and the arterial catheter system may include a second extension tube in fluid communication with the fluid pathway. In some embodiments, a fluidic resistance within the second extension tube may be the second fluidic resistance. In some embodiments, the geometric factor G_{f} of the first extension tube may be different than a geometric factor G_{f} of the second extension tube. In some embodiments, blood may be collected through the first extension tube, and the geometric factor G_{f} of the first extension tube may be greater than a particular portion of the fluid pathway through which the blood for collection does not flow. In some embodiments, the arterial catheter system may include a blood pressure monitoring port between the first extension tube and the second extension tube and/or a blood collection port at a proximal end of first extension tube proximal to the second extension tube.

In some embodiments, the extension tube may include no more than one lumen extending therethrough. In some embodiments, the catheter hub may include a side port disposed between the distal end of the catheter hub and the proximal end of the catheter hub. In some embodiments, the distal end of the extension tube may be integrated with the side port. In some embodiments, the arterial catheter system may include an adapter integrated with the proximal end of the extension tube. In some embodiments, the adapter may include a blood collection port, which may include a female luer.

In some embodiments, the second extension tube may include a distal end and the proximal end. In some embodiments, the arterial catheter system may include a distal adapter and a proximal adapter. In some embodiments, the catheter hub may include a side port disposed between the distal end of the catheter hub and the proximal end of the catheter hub. In some embodiments, the distal end of the second extension tube may be integrated with the side port. In some embodiments, the proximal end of the second extension tube may be integrated into the distal adapter. In some embodiments, the distal end of the first extension tube may be coupled to the distal adapter. In some embodiments, the proximal end of the first extension tube may be coupled to the proximal adapter. In some embodiments, the distal adapter may include a blood pressure monitoring port. In some embodiments, the proximal adapter may include a blood collection port. In some embodiments, the second extension tube may include no more than one lumen extending therethrough.

In some embodiments, the distal end of the second extension tube may be integrated with the side port. In some embodiments, the arterial catheter system may include a distal adapter integrated with the proximal end of the second extension tube and a distal end of the first extension tube. In some embodiments, the arterial catheter system may include a proximal adapter integrated with the proximal end of the first extension tube. In some embodiments, the first extension tube may include a spiral shape. In some embodiments, the proximal adapter may include a blood collection port.

In some embodiments, the distal end of the first extension tube may be integrated with the side port. In some embodiments, the proximal end of the first extension tube may be integrated into the distal adapter. In some embodiments, the distal end of the second extension tube may be coupled to the distal adapter. In some embodiments, the proximal end of the second extension tube may be coupled to the proximal adapter. In some embodiments, the first extension tube may be shorter than the second extension tube. In some embodiments, the distal adapter may include a blood collection port.

In some embodiments, a method of blood collection may include inserting the arterial catheter of the arterial catheter system into an artery of a patient. In some embodiments, the method of blood collection may include collecting arterial blood in a blood collection device coupled to the catheter assembly, whereby the arterial blood flows through the portion of the fluid pathway and into the blood collection device. In some embodiments, the blood collection device may include a vacuum tube. In some embodiments, collecting arterial blood in the blood collection device coupled to the catheter assembly may include inserting a secondary catheter through the distal adapter, the first extension tube, the arterial catheter, and into the artery.

In some embodiments, a method of manufacturing the arterial catheter system may include coupling the catheter assembly to the needle assembly. In some embodiments, the catheter assembly may include the catheter hub and the arterial catheter extending distally from the catheter hub. In some embodiments, the method of manufacturing may include coupling the extension tube to the catheter assembly such that the extension tube is in fluid communication with the catheter assembly and a fluid pathway extends through the arterial catheter, the catheter hub, and the extension tube.

In some embodiments, the method of manufacturing may include selecting the length L of the extension tube and the inner diameter D of the extension tube such that the first fluidic resistance within a portion of the fluid pathway is greater than the second fluidic resistance within the fluid pathway distal to or proximal to the portion of the fluid pathway. In some embodiments, the geometric factor G_{f} of the portion of the fluid pathway within the extension tube equals L/D⁴.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example arterial catheter system, illustrating an example adapter, according to some embodiments;

FIG. 1B is a cross-sectional view of the arterial catheter system, illustrating an example needle assembly removed, according to some embodiments;

FIG. 1C is a cross-sectional view of an example extension tube along the line 1C-1C of FIG. 1A, according to some embodiments;

FIG. 2 is an upper perspective view of the arterial catheter system of FIG. 1, illustrating another example adapter, according to some embodiments;

FIG. 3 is an upper perspective view of the arterial catheter system, illustrating two example extension tubes, according to some embodiments;

FIG. 4 is an upper perspective view of the arterial catheter system, illustrating an example spiral or coiled extension tube, according to some embodiments;

FIG. 5 is an upper perspective view of the arterial catheter system, illustrating two example extension tubes having different lengths and the needle assembly removed, according to some embodiments; and

FIG. 6 is an upper perspective view of the arterial catheter system, illustrating two example extension tubes having different lengths and an example secondary catheter, according to some embodiments.

### DESCRIPTION OF EMBODIMENTS

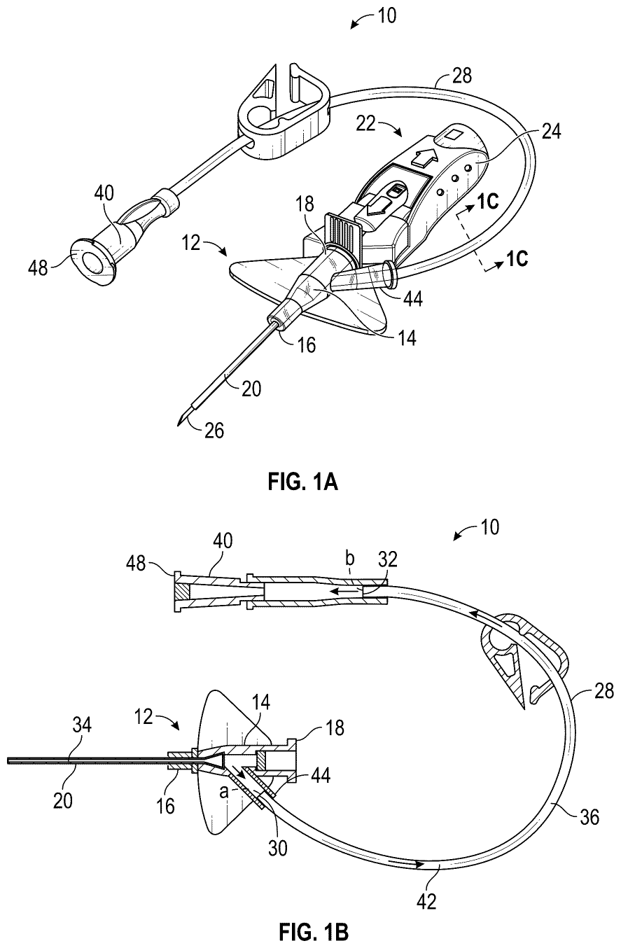

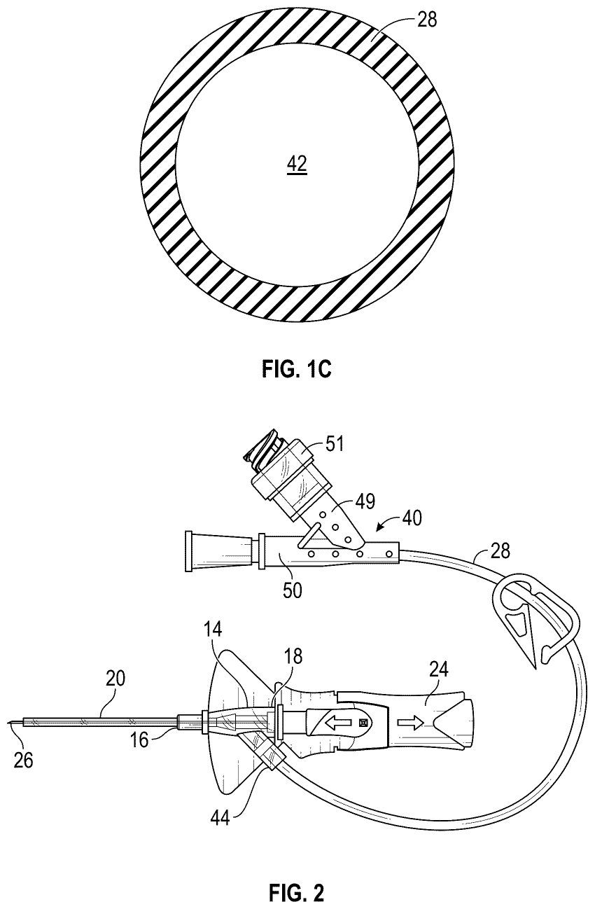

Referring now to FIGS. 1-2, an arterial catheter system 10 is illustrated, according to some embodiments. In some embodiments, the arterial catheter system 10 may be configured to reduce a maximum shear stress on arterial blood drawn from an artery into the arterial catheter system 10 and thereby reduce a risk of hemolysis of the arterial blood, providing an improved blood sample. In some embodiments, the arterial catheter system 10 may include a catheter assembly 12, which may include a catheter hub 14. In some embodiments, the catheter hub 14 may include a distal end 16 and a proximal end 18. In some embodiments, the catheter assembly 12 may include an arterial catheter 20 extending from the distal end 16 of the catheter hub 14. In some embodiments, the arterial catheter 20 may be shorter and/or more rigid than an intravenous catheter, such as a peripheral intravenous catheter.

In some embodiments, the arterial catheter system 10 may include a needle assembly 22, which may include a needle hub 24 and an introducer needle 26. In some embodiments, the arterial catheter system 10 may include an extension tube 28 coupled to the catheter assembly 12 and having a distal end 30 and a proximal end 32. In some embodiments, the arterial catheter system 10 may include a fluid pathway 34 extending through at least the arterial catheter 20, catheter hub 14, and the extension tube 28.

In some embodiments, a geometric factor G_{f} of a portion 36 of the fluid pathway 34 within the extension tube 28 may equal L/D⁴, wherein L is the length of the extension tube 28 and D is the inner diameter of the portion of the fluid pathway 34 within the extension tube 28. In these embodiments, the portion 36 of the fluid pathway 34 may be cylindrical along an entirety of the length L, and the inner diameter D may be constant along the length L. The length L corresponds to an entire length of the extension tube 28 or a length from a to b, according to some embodiments. In some embodiments, the geometric factor G_{f} of the portion 36 of the fluid pathway 34 within the extension tube 28 may be defined such that a fluidic resistance

$${R}_{f} = \frac{128 μ}{π} {G}_{f} ,$$

where

$${G}_{f} = \frac{L}{{D}^{4}} .$$

In some embodiments, the geometric factor G_{f} of the portion 36 of the fluid pathway 34 may be selected to reduce a maximum shear stress on arterial blood drawn from an artery and thereby reduce a risk of hemolysis of the arterial blood.

In some embodiments, the arterial catheter 20 may be a 20 G arterial catheter. In these embodiments, the length L and the inner diameter D may be selected such that the geometric factor G_{f} of the portion of the fluid pathway within the extension tube 28 is 3.41 E+06 (1/in³) or higher. In some embodiments, the length L and the inner diameter D for a 20 G arterial catheter may be selected such that the geometric factor G_{f} of the portion 36 of the fluid pathway 34 within the extension tube 28 is 3.41 E+06 (1/in³)+/−10%.

In some embodiments, the arterial catheter 20 may be a 18 G arterial catheter. In these embodiments, the length L and the inner diameter D may be selected such that the geometric factor G_{f} of the portion of the fluid pathway within the extension tube 28 is 2.88 E+06 (1/in³) or higher. In some embodiments, the length L and the inner diameter D for a 18 G arterial catheter may be selected such that the geometric factor G_{f} of the portion 36 of the fluid pathway 34 within the extension tube 28 is 2.88 E+06 (1/in³)+/−10%.

In some embodiments, the arterial catheter 20 may be a 22 G arterial catheter. In these embodiments, the length L and the inner diameter D may be selected such that the geometric factor G_{f} of the portion of the fluid pathway within the extension tube 28 is 1.05 E+07 (1/in³) or higher. In some embodiments, the length L and the inner diameter D for a 22 G arterial catheter may be selected such that the geometric factor G_{f} of the portion 36 of the fluid pathway 34 within the extension tube 28 is 1.05 E+07 (1/in³)+/−10%.

In some embodiments, the arterial catheter 20 may be a 24 G arterial catheter. In these embodiments, the length L and the inner diameter D may be selected such that the geometric factor G_{f} of the portion of the fluid pathway within the extension tube 28 is 3.20 E+07 (1/in³) or higher. In some embodiments, the length L and the inner diameter D for a 24 G arterial catheter may be selected such that the geometric factor G_{f} of the portion 36 of the fluid pathway 34 within the extension tube 28 is 3.20 E+07 (1/in³)+/−10%.

In some embodiments, the fluidic resistance may be a first fluidic resistance. In some embodiments, the first fluidic resistance of the portion 36 of the fluid pathway 34 within the extension tube 28 may be greater than a second fluidic resistance within the fluid pathway 34 distal to the portion 36 of the fluid pathway 34. For example, the first fluidic resistance may be greater than a particular fluidic resistance within a lumen 38 of the catheter hub 14, where arterial blood may travel prior to reaching the extension tube 28 and adapter 40, which may be coupled to a blood collection device for blood collection.

A blood cell experiences shear stress as it flows in a fluid pathway. The maximum shear stress is along the wall of the fluid pathway, or wall shear stress. Wall shear stress on blood cells is considered a major source of mechanical damage to blood cells. For a cylindrical fluid path, the wall shear stress is typically expressed as:

$$τ = \frac{1}{2} \cdot \frac{Δ p}{L} \cdot ( kr )$$

in which ΔP is the pressure drop along a path with a length of L and an interior radius of r. k is shrinkage index.

To fill a certain volume of collection tube, V, with a flow rate of Q, the time needed can be simply assessed by:

$$t = \frac{v}{ℚ} = 8 μ v \cdot \frac{1}{π {r}^{4}} / ( \frac{Δ p}{L} )$$

in which μ is the dynamic viscosity of the fluid. Hemolysis is typically associated with both the wall shear stress and the time a blood cell is exposed to wall shear stress. From literature, it has been widely considered that hemolysis index can be approached as a function of:

$$HI ( \% ) = A * {t}^{α} * {τ}^{β}$$

in which A, α, and β are coefficients.

In principle, the hemolysis index is related to pressure gradient and cross-sectional characteristic dimension:

$$HI ( \% ) ∝ {( \frac{Δ P}{l} )}^{β - a} \cdot {( \frac{1}{r} )}^{4 α - β}$$

Fluid flow in a particular extension tube with a cylindrical fluid pathway therethrough can be analyzed using Poiseuille's equation:

$$Q = \frac{π {D}^{4} Δ P}{128 μ L} = \frac{Δ P}{{R}_{f}}$$

where ΔP is a change in pressure gradient across the length of the extension tube, D and L are the inner diameter and length, respectively, of the cylindrical fluid pathway through the particular extension tube, μ is the viscosity of a fluid, and

$${R}_{f} = \frac{128 μ L}{π {D}^{4}}$$

is the fluidic resistance. The particular extension tube may include or correspond to the extension tube 28. Since μ is the viscosity of the fluid and not part of the extension tube geometry, the geometric factor G_{f} is defined such that R_{f} (the fluidic resistance) is

$${R}_{f} = \frac{128 μ}{π} {G}_{f} ,$$

where

$${G}_{f} = \frac{L}{{D}^{4}} .$$

In some embodiments, the extension tube 28 may have multiple sections with lengths (L1, L2, L3) and inner diameters of (D1, D2, D3), the geometric factor is then:

$${G}_{f} = \frac{L 1}{D {1}^{4}} + \frac{L 2}{D {2}^{4}} + \frac{L 3}{D {3}^{4}}$$

In some embodiments, the extension tube 28 may have an inner diameter that changes over the length of the extension tube, the geometric factor is then:

$${G}_{f} = {\int }_{0}^{L} \frac{dl}{{D ( l )}^{4}}$$

In some embodiments, the extension tube 28 may have a cross section that is not circular. In this case, the geometric factor can be determined by measuring the flow rate (Q) at given pressure (ΔP) with known viscosity (μ) fluid:

$${G}_{f} = \frac{π Δ P}{128 μ Q}$$

In some embodiments, the first fluidic resistance, which may be lower than the second fluidic resistance, may facilitate a decreased flow rate of arterial blood within the portion 36 of the fluid pathway 34 within the extension tube 28 such that the maximum shear stress is reduced within the portion 36 of the fluid pathway 34 and there is a decreased risk of hemolysis of the arterial blood that will be collected. In these embodiments, the length L and the inner diameter D of the extension tube 28, which may determine the geometric factor G_{f} of the portion 36 of the fluid pathway 34, may be selected to increase the first fluidic resistance and decrease the flow rate within the portion 36 of the fluid pathway such that the risk of hemolysis is decreased but the flow rate is still adequate for blood collection.

In some embodiments, the extension tube 28 may include no more than one lumen 42 extending therethrough. In these embodiments, a single lumen may be sufficient for the extension tube 28 in the arterial catheter system 10 since arterial catheters are rarely used for infusion where an extension tube with higher fluidic resistance might reduce the infusion rate significantly. In some embodiments, the extension tube 28 may include more than one lumen. In some embodiments, the catheter hub 14 may include a side port 44 disposed between the distal end 16 of the catheter hub 14 and the proximal end 18 of the catheter hub 14. In some embodiments, the distal end 30 of the extension tube 28 may be coupled to or integrated with the side port 44. In some embodiments, the arterial catheter system 10 may include an adapter 40 coupled to or integrated with the proximal end 32 of the extension tube 28. In some embodiments, the adapter 40 may include a blood collection port 48, which may include a female luer. In some embodiments, a blood collection device may be coupled to the blood collection port 48. In some embodiments, the blood collection device may be coupled to the blood collection port 48 via a needleless access connector disposed between the blood collection device and the blood collection port 48 and/or directly coupled to both.

As illustrated in FIG. 2, in some embodiments, the adapter 40 may include a Y-connector, which may include a blood collection port 49 configured to couple to a blood collection device and/or a blood pressure monitoring port 50 configured to couple to an arterial pressure monitor. In some embodiments, the blood collection port 49 and/or the blood pressure monitoring port 50 may include a female luer. In some embodiments, a needleless connector 51 may be coupled to a blood collection port 49 and/or the blood collection device may be coupled to the needleless connector 51. In some embodiments, the blood collection device may be coupled to the blood collection port 49 via the needleless connector 51 disposed between the blood collection device and the blood collection port 49 and/or directly coupled to both. In some embodiments, the arterial pressure monitor may be coupled to the blood pressure monitoring port 50.

In some embodiments, the blood collection device may include or correspond to a blood collection container. In some embodiments, the blood collection container may include a syringe, an evacuated blood collection tube (or vacuum tube), a small sample collection device, or any other container configured to collect blood from a patient via a pressure differential. In some embodiments, the blood collection device may include a luer lock access device, such as, for example, the VACUTAINER® LUER-LOK™ ACCESS DEVICE available from Becton Dickinson & Company. In some embodiments, the luer lock access device may include a blood collection tube holder further described, for example, in U.S. patent application Ser. No. 17/075,420, filed Oct. 20, 2020, entitled “BLOOD COLLECTION SYSTEM WITH USER-ADJUSTED PRESSURE MANAGEMENT AND RELATED METHODS,” which is incorporated by reference in its entirety.

In some embodiments, the blood collection device may include an instrument delivery device configured to deliver a secondary catheter through a catheter assembly. In some embodiments, the instrument delivery device may be further described in U.S. Pat. No. 11,969,247, granted Apr. 30, 2024, entitled “EXTENSION HOUSING A PROBE OR INTRAVENOUS CATHETER,” U.S. patent application Ser. No. 16/388,650, filed Apr. 18, 2019, entitled “INSTRUMENT DELIVERY DEVICE HAVING A ROTARY ELEMENT,” U.S. Pat. No. 11,173,277, granted Nov. 16, 2021, entitled “MULTI-DIAMETER CATHETER AND RELATED DEVICES AND METHODS,” U.S. Pat. No. 11,406,795, filed Aug. 9, 2022, entitled “DELIVERY DEVICE FOR A VASCULAR ACCESS INSTRUMENT,” U.S. Pat. No. 11,337,628, granted May 24, 2022, entitled “SYRINGE-BASED DELIVERY DEVICE FOR A VASCULAR ACCESS INSTRUMENT,” U.S. Pat. No. 11,547,832, granted Jan. 10, 2023, entitled “CATHETER DELIVERY DEVICE AND RELATED SYSTEMS AND METHODS,” and U.S. Pat. No. 11,504,503, granted Nov. 22, 2022, entitled “VASCULAR ACCESS INSTRUMENT HAVING A FLUID PERMEABLE STRUCTURE AND RELATED DEVICES AND METHODS,” which are incorporated by reference in their entirety.

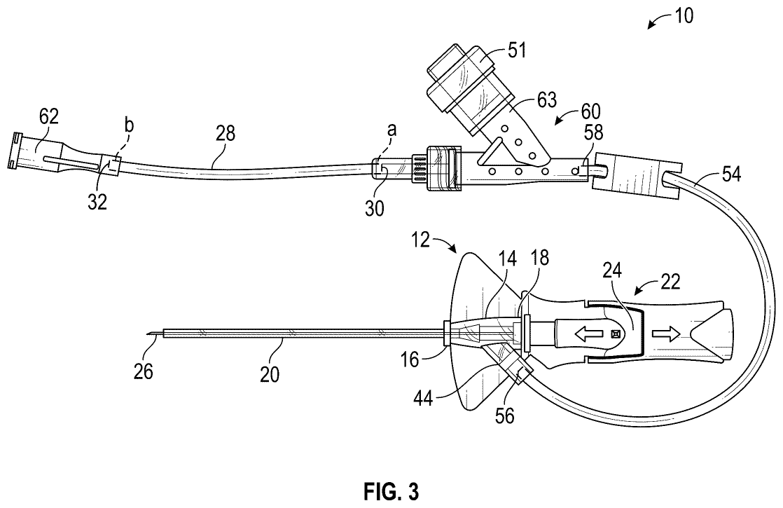

Referring now to FIG. 3, in some embodiments, the extension tube 28 may be a first extension tube, and the arterial catheter system 10 may include a second extension tube 54 in fluid communication with the fluid pathway 34. In some embodiments, a fluidic resistance within the second extension tube 54 may be the second fluidic resistance. In some embodiments, the geometric factor G_{f} of the first extension tube may be different than a geometric factor G_{f} of the second extension tube 54. In some embodiments, the first fluidic resistance of the portion 36 of the fluid pathway 34 within the extension tube 28 (see FIG. 1B, for example) may be lower than the second fluidic resistance.

In some embodiments, the first extension tube and/or the second extension tube 54 may be flexible, which may ease of use of the arterial catheter system 10. In other embodiments, the first extension tube and/or the second extension tube 54 may be rigid or semi-rigid. In some embodiments, the first extension tube and/or the second extension tube 54 may be constructed of plastic. In some embodiments, any suitable first lumen may be substituted for the first extension tube and/or any suitable second lumen may be substituted for the second extension tube 54.

In some embodiments, the second extension tube 54 may include a distal end 56 and a proximal end 58. In some embodiments, the arterial catheter system 10 may include a distal adapter 60 and a proximal adapter 62. In some embodiments, the catheter hub 14 may include the side port 44 disposed between the distal end 16 of the catheter hub 14 and the proximal end 18 of the catheter hub 14. In some embodiments, the distal end 56 of the second extension tube 54 may be coupled to or integrated with the side port 44. In some embodiments, the proximal end 58 of the second extension tube 54 may be coupled to or integrated into the distal adapter 60. In some embodiments, the distal end 30 of the first extension tube may be coupled to the distal adapter 60. In some embodiments, the proximal end 32 of the first extension tube may be coupled to the proximal adapter 62. In some embodiments, the distal adapter 60 may include a blood pressure monitoring port 63.

In some embodiments, the proximal adapter 62 may include a blood collection port. In some embodiments, the blood collection port and/or the blood pressure monitoring port 63 may include a female luer. In some embodiments, the first extension tube may be proximal to the second extension tube 54 and the blood collection port may be disposed at the proximal end 32 of the first extension tube.

In some embodiments, the arterial catheter system 10 may include the blood pressure monitoring port 63 between the first extension tube and the second extension tube 54, which both may be coupled to or integrated with the distal adapter 60. Thus, in some embodiments, arterial blood for pressure testing may travel through the second extension tube 54 but not the first extension tube to provide an accurate arterial blood pressure reading.

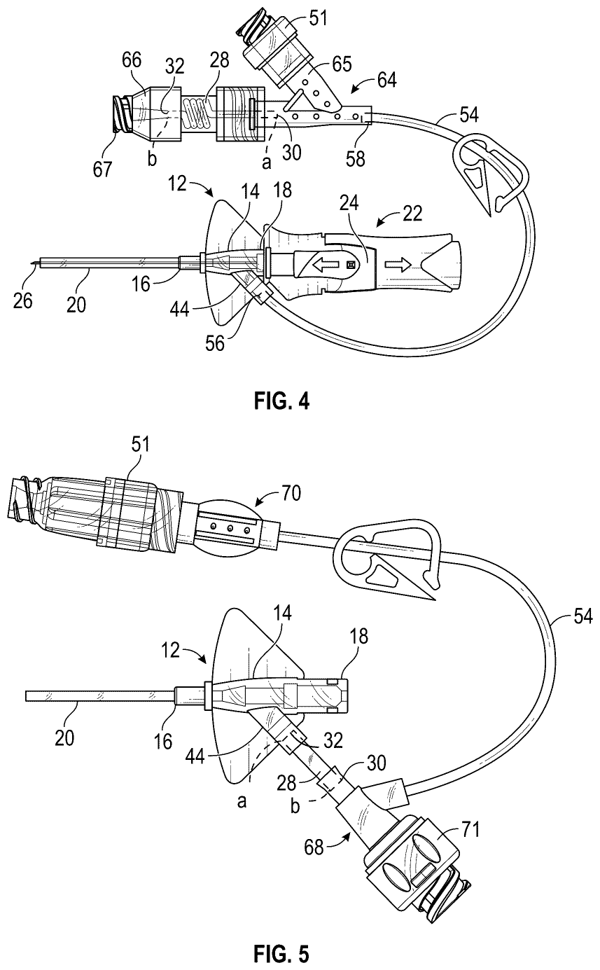

Referring now to FIG. 4, in some embodiments, the distal end 56 of the second extension tube 54 may be coupled to or integrated with the side port 44. In some embodiments, the arterial catheter system 10 may include a distal adapter 64 coupled to or integrated with the proximal end 58 of the second extension tube 54 and a distal end 30 of the first extension tube (corresponding to extension tube 28). In some embodiments, the arterial catheter system 10 may include a proximal adapter 66 coupled to or integrated with the proximal end 32 of the first extension tube. In some embodiments, the first extension tube may include a spiral shape, which may facilitate a compact device for easy use by a clinician. In some embodiments, the distal adapter 64 may include a blood pressure monitoring port 65. In some embodiments, the proximal adapter 66 may include a blood collection port 67. In some embodiments, the blood collection port 67 and/or the blood pressure monitoring port 65 may include a female luer.

Referring now to FIG. 5, in some embodiments, the distal end 16 of the first extension tube (corresponding to extension tube 28) may be coupled to or integrated with the side port 44. In some embodiments, the proximal end 18 of the first extension tube may be coupled to or integrated into a distal adapter 68. In some embodiments, the distal end 56 of the second extension tube 54 may be coupled to the distal adapter 68. In some embodiments, the proximal end 58 of the second extension tube 54 may be coupled to a proximal adapter 70. In some embodiments, the first extension tube may be shorter than the second extension tube 54. In some embodiments, the distal adapter 68 may include a blood collection port 71 and/or the proximal adapter 70 may include a blood pressure monitoring port. In some embodiments, the blood collection port 71 and/or the blood pressure monitoring port may include a female luer. In some embodiments, the geometric factor G_{f} of the first extension tube may be higher than a geometric factor G_{f} of the second extension tube 54, because the first extension tube may be used for blood collection.

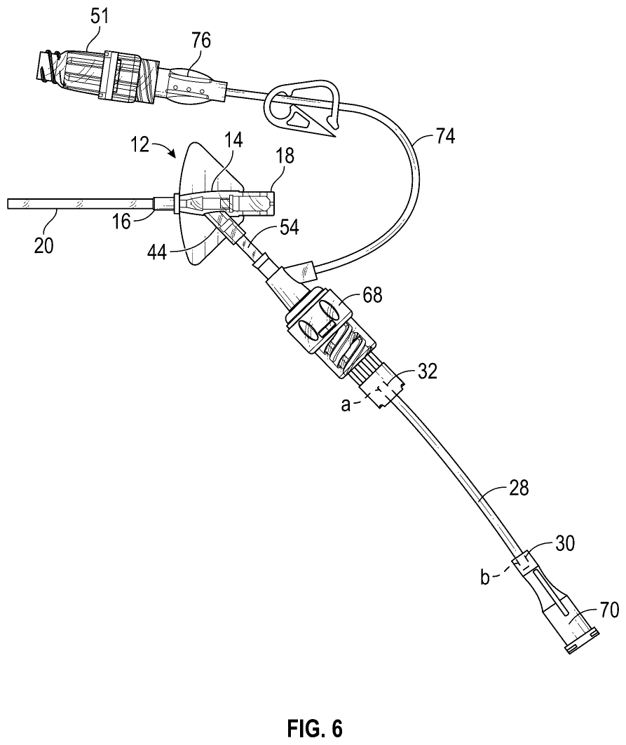

Referring now to FIG. 6, in some embodiments, the distal end 16 of the first extension tube (corresponding to extension tube 28) may be coupled to or integrated with the distal adapter 68. In some embodiments, the proximal end 18 of the first extension tube may be coupled to or integrated into the proximal adapter 70. In some embodiments, the distal end 56 of the second extension tube 54 may be coupled to the side port 44. In some embodiments, the proximal end 58 of the second extension tube 54 may be coupled to the distal adapter 68. In some embodiments, the second extension tube 54 may be shorter than the first extension tube. In some embodiments, the proximal adapter 70 may include a blood collection port. In some embodiments, a third extension tube 74 may extend from the distal adapter 68. A blood pressure monitoring port 76 and/or a needleless connector 51 may be disposed at a proximal end of the third extension tube 74. In some embodiments, the geometric factor G_{f} of the first extension tube and a geometric factor G_{f} of the second extension tube 54 may be higher than a geometric factor G_{f} of the third extension tube 74, because the first extension tube and the second extension tube 54 may be used for blood collection.

Referring now to FIGS. 1-6, in some embodiments, a method of blood collection may include inserting the arterial catheter 20 of the arterial catheter system 10 into an artery of a patient. In some embodiments, the method of blood collection may include collecting arterial blood in the blood collection device, which may be coupled to the catheter assembly 12, whereby the arterial blood flows through the portion 36 of the fluid pathway 34 (see FIG. 1B, for example) and into the blood collection device. In some embodiments, the blood collection device may include a vacuum tube. Referring to FIGS. 5-6, in some embodiments, collecting arterial blood in the blood collection device coupled to the catheter assembly 12 may include inserting the secondary catheter through the distal adapter 68, the first extension tube, the arterial catheter 20, and into the artery. In some embodiments, the distal adapter 68 may provide near-port access to the arterial catheter system 10, which may allow a length of the secondary catheter or other blood collection device inserted through the catheter assembly 12 to be shorter.

Referring again to FIGS. 1-6, in some embodiments, a method of manufacturing the arterial catheter system 10 may include coupling the catheter assembly 12 to the needle assembly 22. In some embodiments, the catheter assembly 12 may include the catheter hub 14 and the arterial catheter 20 extending distally from the catheter hub 14. In some embodiments, the method of manufacturing may include coupling the extension tube 28 to the catheter assembly 12 such that the extension tube 28 is in fluid communication with the catheter assembly 12 and the fluid pathway 34 extends through the arterial catheter 20, the catheter hub 14, and the extension tube 28.

In some embodiments, the method of manufacturing may include selecting the length L of the extension tube 28 and the inner diameter D of the extension tube 28 such that the first fluidic resistance within a portion of the fluid pathway 34 is greater than the second fluidic resistance within the fluid pathway distal to or proximal to the portion of the fluid pathway. In some embodiments, the geometric factor G_{f} of the portion of the fluid pathway within the extension tube equals L/D⁴.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

We claim:

1. An arterial catheter system, comprising:
a catheter assembly, comprising:
a catheter hub, comprising a distal end and a proximal end;
an arterial catheter extending from the distal end of the catheter hub;
a needle assembly, comprising:
a needle hub;
an introducer needle;
an extension tube coupled to the catheter assembly and having a distal end and a proximal end; and
a fluid pathway extending through the arterial catheter, catheter hub, and the extension tube,
wherein a first fluidic resistance within a portion of the fluid pathway within the extension tube is greater than a second fluidic resistance within the fluid pathway distal to or proximal to the portion of the fluid pathway.

2. The arterial catheter system of claim 1, wherein the extension tube is a first extension tube, wherein the arterial catheter system comprises a second extension tube in fluid communication with the fluid pathway, wherein a geometric factor G_{f} of the portion of the fluid pathway within the first extension tube is different than a geometric factor Gr of another portion of the fluid pathway within the second extension tube, wherein the first extension tube is proximal to the second extension tube and a blood collection port is disposed at a proximal end of the first extension tube.

3. The arterial catheter system of claim 1, wherein the extension tube comprises no more than one lumen extending therethrough.

4. The arterial catheter system of claim 1, wherein the catheter hub further comprises a side port disposed between the distal end of the catheter hub and the proximal end of the catheter hub, wherein the distal end of the extension tube is integrated with the side port, further comprising an adapter integrated with the proximal end of the extension tube, wherein the adapter comprises a blood collection port, wherein the blood collection port comprises a female luer.

5. The arterial catheter system of claim 1, wherein the extension tube is a first extension tube, wherein the arterial catheter system further comprises a second extension tube having a distal end and a proximal end, wherein the arterial catheter system further comprises a distal adapter and a proximal adapter, wherein the catheter hub further comprises a side port disposed between the distal end of the catheter hub and the proximal end of the catheter hub, wherein the distal end of the second extension tube is integrated with the side port, wherein the proximal end of the second extension tube is integrated into the distal adapter, wherein the distal end of the first extension tube is coupled to the distal adapter, wherein the proximal end of the first extension tube is coupled to the proximal adapter.

6. The arterial catheter system of claim 5, wherein the distal adapter comprises a blood pressure monitoring port, wherein the proximal adapter comprises a blood collection port.

7. The arterial catheter system of claim 5, wherein the first extension tube comprises no more than one lumen extending therethrough, wherein the second extension tube comprises no more than one lumen extending therethrough.

8. The arterial catheter system of claim 1, wherein the extension tube is a first extension tube, wherein the arterial catheter system comprises a second extension tube having a distal end and a proximal end, wherein the catheter hub further comprises a side port disposed between the distal end of the catheter hub and the proximal end of the catheter hub, wherein the distal end of the second extension tube is integrated with the side port, wherein the arterial catheter system further comprises a distal adapter integrated with the proximal end of the second extension tube and a distal end of the first extension tube, wherein the arterial catheter system further comprises a proximal adapter integrated with the proximal end of the first extension tube, wherein the first extension tube comprises a spiral shape, wherein the proximal adapter comprises a blood collection port.

9. The arterial catheter system of claim 1, wherein the extension tube is a first extension tube, wherein the arterial catheter system further comprises a second extension tube having a distal end and a proximal end, wherein the arterial catheter system further comprises a distal adapter and a proximal adapter, wherein the catheter hub further comprises a side port disposed between the distal end of the catheter hub and the proximal end of the catheter hub, wherein the distal end of the first extension tube is integrated with the side port, wherein the proximal end of the first extension tube is integrated into the distal adapter, wherein the distal end of the second extension tube is coupled to the distal adapter, wherein the proximal end of the second extension tube is coupled to the proximal adapter, wherein the first extension tube is shorter than the second extension tube, wherein the distal adapter comprises a blood collection port.

10. A method of blood collection, comprising:
inserting an arterial catheter of an arterial catheter system into an artery of a patient, wherein the arterial catheter system comprises:
a catheter assembly, comprising:
a catheter hub, comprising a distal end and a proximal end;
an arterial catheter extending from the distal end of the catheter hub;
a needle assembly, comprising:
a needle hub;
an introducer needle;
an extension tube coupled to the catheter assembly and having a distal end and a proximal end; and
a fluid pathway extending through the arterial catheter, catheter hub, and the extension tube,
wherein a first fluidic resistance within a portion of the fluid pathway within the extension tube is greater than a second fluidic resistance within the fluid pathway distal to or proximal to the portion of the fluid pathway; and
collecting arterial blood in a blood collection device coupled to the catheter assembly, whereby the arterial blood flows through the portion of the fluid pathway and into the blood collection device.

11. The method of claim 10, wherein the blood collection device comprises a vacuum tube.

12. The method of claim 10, wherein the extension tube is a first extension tube, wherein the arterial catheter system further comprises a second extension tube having a distal end and a proximal end, wherein the arterial catheter system further comprises a distal adapter and a proximal adapter, wherein the catheter hub further comprises a side port disposed between the distal end of the catheter hub and the proximal end of the catheter hub, wherein the distal end of the first extension tube is integrated with the side port, wherein the proximal end of the first extension tube is integrated into the distal adapter, wherein the distal end of the second extension tube is coupled to the distal adapter, wherein the proximal end of the second extension tube is coupled to the proximal adapter, wherein the first extension tube is shorter than the second extension tube, wherein the distal adapter comprises a blood collection port;
wherein collecting arterial blood in the blood collection device coupled to the catheter assembly comprises inserting a secondary catheter through the distal adapter, the first extension tube, the arterial catheter, and into the artery.

13. A method of manufacturing an arterial catheter system, the method comprising:
coupling a catheter assembly to a needle assembly, wherein the catheter assembly comprises a catheter hub and an arterial catheter extending distally from the catheter hub;
coupling an extension tube to the catheter assembly such that the extension tube is in fluid communication with the catheter assembly and a fluid pathway extends through the arterial catheter, the catheter hub, and the extension tube; and
selecting a length L of the extension tube and an inner diameter D of the extension tube such that a first fluidic resistance within a portion of the fluid pathway is greater than a second fluidic resistance within the fluid pathway distal to or proximal to the portion of the fluid pathway.

14. The method of claim 13, wherein the catheter is a 20 G catheter, wherein the length L and the inner diameter D are selected such that a geometric factor G_{f} of the portion of the fluid pathway of the extension tube is 3.33 E+06 (1/in³) or higher.
